SEQUENCE LISTING (1 sequence)

SEQ ID NO: 1 - protein, 70 aa, Homo sapiens
EAEEDGDLQCLCVKTTSQVRPRHITSLEVIKAGPHCPTAQLIATLKNGRKICLDLQAPLYKKIIKKLLES

(19) United States
(12) Patent Application Publication
(10) Pub. No.: US 2026/0023088 A1
(43) Pub. Date: Jan. 22, 2026

(54) REAGENT AND METHOD FOR DIAGNOSING THROMBOTIC EVENTS

(71) Applicants: Biokit Research & Development,S.L.U, LLIÇÀ D'AMUNT (ES); Universitätsmedizin Greifswald, Greifswald (DE)

(72) Inventors: Olga Esteban Tortajada, Barcelona (ES); Andreas Greinacher, Suderholz (DE); Roser Llevadot, Barcelona (ES); Paulina Dobosz, Lublin (PL); Konstanze Aurich, Greifswald (DE); Marta Broto Aviles, Canovelles (ES)

(73) Assignees: Biokit Research & Development,S.L.U, Barcelona (ES); Universitätsmedizin Greifswald, Greifswald (DE)

(21) Appl. No.: 18/874,783

(22) PCT Filed: May 11, 2023
(86) PCT No.: PCT/EP2023/062615

§ 371 Date: Dec. 13, 2024

(30) Foreign Application Priority Data
Jul. 1, 2022 (ES) 22382635.5

(51) Int. Cl. G01N 33/86; G01N 33/543

(52) U.S. Cl. CPC G01N 33/86; G01N 33/54326; G01N 33/54346; G01N 2800/226; G01N 2800/52

(57) ABSTRACT

The disclosure refers to reagents, kits and methods for detecting anti-PF4 antibodies and diagnosing thrombotic events not induced by heparin, including vaccine-induced thrombotic thrombocytopenia. The method provided by the disclosure comprises: (i) contacting whole blood. plasma or serum sample obtained from a subject with a reagent comprising: (a) a binding molecule selected from the group consisting of platelet factor 4 protein (PF4), a fragment of PF4 which can bind anti-PF4 antibodies, and an anti-idiotype antibody of anti-platelet factor 4 antibodies (anti-PF4 antibodies); and (b) a solid support, wherein the binding molecule (a) is covalently bound to the surface of the solid support (b), and wherein the binding molecule (a) does not include heparin or a heparin surrogate, and (ii) analysing the sample to detect a complex formed by the reagent and the anti-PF4 antibodies, wherein detecting the complex is indicative of the sample containing anti-PF4 antibodies.

This application claims the benefit of European Patent Application no. EP22382635.5 filed Jul. 1, 2022.

### TECHNICAL FIELD

The present invention relates to the field of clinical diagnosis, in particular to the determination of anti-platelet factor 4 (anti-PF4) antibodies, which allows, for example, diagnosis of non-heparin related thrombotic thrombocytopenias, such as vaccine-induced thrombotic thrombocytopenia (VITT).

### BACKGROUND ART

Vaccination against severe acute respiratory syndrome coronavirus-2 (SARS-COV-2) is the most important countermeasure to fight the ongoing Covid-19 pandemic. One of the first vaccines approved by the European Medicines Agency was recombinant chimpanzee adenoviral [ChAdOx1-S] vector encoding the spike glycoprotein of SARS-COV-2, COVID-19 Vaccine AstraZeneca [also known as ChAdOx1 nCOV-19, AZD1222, Vaxzevria], which is an adenoviral vector-based vaccine.

Unfortunately, cases of cerebral venous sinus thrombosis (CVST) and cases of splanchnic vein thrombosis in combination with moderate to severe thrombocytopenia have been reported in several countries in healthy individuals after ChAdOx1 nCov-19 vaccination. This novel disorder, “vaccine-induced immune thrombotic thrombocytopenia (VITT)”, is associated with high titers of immunoglobulin G class antibodies directed against the cationic platelet chemokine, platelet factor 4 (PF4; CXCL4). These antibodies potently activate platelets via platelet FcγIIa receptors, with platelet activation greatly enhanced by PF4. Another adenovirus-based vaccine, COVID-19 Vaccine Janssen (Johnson& & Jonson), also appears to be associated with formation of anti-PF4 antibodies.

PF4 opsonizes polyanionic surfaces of pathogens, facilitating the binding of anti-PF4 antibodies. This is likely an evolutionary old immune defense mechanism, as anti-PF4 producing B-cells can be found in nearly all individuals, including even in newborn cord blood. However, a strong anti-PF4 antibody response, when misdirected, underlies the thromboembolic disorder, immune heparin-induced thrombocytopenia and its most severe presentation, autoimmune heparin-induced thrombocytopenia. Autoimmune heparin-induced thrombocytopenia is characterized by formation of platelet-activating anti-PF4 antibodies with very high avidity that are reactive even in the absence of heparin. Severe heparin-induced thrombocytopenia also features “pancellular” activation (platelets, neutrophils, monocytes, endothelium). VITT closely mimics autoimmune heparin-induced thrombocytopenia (HIT) both clinically and serologically (Greinacher et al, “Thrombotic Thrombocytopenia after ChAdOx1 nCov-19 Vaccination”. DOI:

10.1056/NEJMoa2104840). One of the major risk factors for formation of anti-PF4 antibodies is inflammation and tissue trauma, which substantially increase the risk for forming pathogenic anti-PF4 antibodies.

Besides VITT, other thrombotic events have been observed which are not related to heparin (Favaloro et al, 2022 “Antibodies against Platelet Factor 4 and Their Associated Pathologies: From HIT/HITT to Spontaneous HIT-Like Syndrome, to COVID-19, to VITT/TTS” DOI: 10.3390/antib1101000). New thrombotic events also include vascular occlusions in the arterial system. The factors causing said conditions are largely unknown but, nevertheless, they share the same difficulty in diagnosis as VITT. As a result, these conditions are under-diagnosed with the current clinical protocols, which mainly include using rapid HIT assays or commercial anti-PF4 ELISA assays and functional assays for heparin dependent antibodies.

Currently, several commercial immunoassays are available for diagnosing HIT, both as classical IgG-specific and polyspecific ELISA assays (e.g., Asserachrom HPIA IgG, Lifecodes PF4 lgG, Hyphen Biomed Zymutest HIA IgG, AESKULISA HIT II) as well as rapid assays (e.g., HemosIL® AcuStar HIT-IgG_{(PF4-H)}, HemosIL® HIT-Ab_{(PF4-H)}, Diamed PaGIA gel, STic Expert). All assays use PF4 protein complexed with heparin or a heparin surrogate, such as polyvinyl sulphate, to detect anti-PF4 antibodies triggered by heparin (anti-PF4/H antibodies) in patients suspected of suffering from HIT. However, said available assays are not appropriate for diagnosing VITT. Previous studies have shown that rapid assays have very poor sensitivity for VITT diagnosis, which renders them unsuitable for diagnosing this particular condition. On the other side, state of the art anti-PF4/heparin or anti-PF4/PVS ELISA assays only detect some VITT cases and no single ELISA method appeared to detect all cases of VITT (Platton et al, “Evaluation of laboratory assays for anti-platelet factor 4 antibodies after ChAdOx1 nCOV-19 vaccination”. DOI: 10.1111/jth. 15362). Most problematic is that even those assays which recognize some VITT-like antibodies also recognize heparin-dependent HIT-like antibodies, such that said assays cannot differentiate between the two antibody specificities. This has prompted experts to recommend that, when an ELISA result is negative, then a second ELISA or a platelet activation assay should be considered where there is strong clinical suspicion of VITT, a routine that significantly complicates the diagnosis. Even this step results in false negative results if the functional test is performed with heparin enhancement instead of PF4 enhancement. Nevertheless, the ELISA assays, which are more widely available in diagnostic laboratories have a long turnaround time of at least 6 to 8 hours and only a small number of laboratories are able to perform platelet activation assays.

An additional problem is that the available immunoassays are designed to measure the presence of anti-PF4/H antibodies and, consequently, they are not specific for anti-PF4 antibodies found in patients with VITT. Therefore, these immunoassays, do not differentiate between HIT and VITT patients (if they detect VITT at all). Thus, in patients receiving heparin, there is no way to differentiate if the thrombotic event has been triggered by heparin or by another reason, such as vaccination or other unknown reasons. This lack of differential diagnosis of VITT with respect to HIT makes it impossible to discern in which patients heparin may be further administered and in which ones heparin administration must be discontinued, often to be substituted by different, far more expensive, antithrombotics.

Moreover, increasingly patients are recognized with a thrombotic disorder independent of the presence of heparin or vaccination. In some of these patients, heparin-dependent and heparin-independent anti-PF4 antibodies, in variable proportions, are identified. Currently, these antibodies can be differentiated by functional assays. New and widely applicable assays that can differentiate among these various anti-PF4 disorders are needed to evaluate systematically the prevalence of these different patient cohorts. This is clinically relevant because anti-PF4 heparin-independent antibodies required adjunctive measures (besides anticoagulation) to deescalate FcγRIIa-dependent cell activation that triggers massive hypercoagulability.

There is therefore an urgent need to provide sensitive and specific assays to detect anti-PF4 heparin independent antibodies for diagnosing VITT and other thrombotic events not induced by heparin or vaccination which are cost-effective, rapid, and easy to implement in the clinical practice.

### SUMMARY

The present inventors have developed a new method for detecting heparin independent anti-PF4 antibodies (from now on also referred to simply as “anti-PF4 antibodies”) which overcomes the drawbacks mentioned above. The method of the invention specifically detects anti-PF4 antibodies but not heparin-dependent HIT-like antibodies. Therefore, the new method allows for diagnosing thrombotic events not induced by heparin, e.g., VITT or other types. The new method is based on a reagent that is comprised by a molecule capable of binding anti-PF4 antibodies from human patients that are suffering or have suffered from such thrombotic event not induced by heparin and a solid support, wherein the molecule capable of binding anti-PF4 antibodies is covalently bound to the surface of the solid support.

Thus, a first aspect of the present disclosure provides a reagent comprising: (a) a molecule for binding anti-PF4 antibodies and (b) a solid support, wherein the molecule is covalently bound to the surface of the solid support.

An illustrative schematic representation of a reagent according to the first aspect of the disclosure is shown if FIG. 1. The covalent bond of the molecule for binding anti-PF4 antibodies (from now on also termed “binding molecule”) to the solid support is important, since the inventors have found that attachment of the binding molecule by other means barely detects anti-PF4 antibodies in a few samples from VITT patients (see FIGS. 4 and 5). It was also found that the binding molecule must not be forming a complex with heparin or other heparin surrogates (such as PVS). This is evidenced in FIG. 3, where it may be seen that plasma from all the VITT patients showed a high signal only when tested using the reagent of the disclosure, which contains PF4 alone as the only binding molecule. Thus, the binding molecule (a) of the reagent of the first aspect of the disclosure does not include heparin or any other heparin surrogate, i.e. the binding molecule (a) is not forming a complex with heparin or a heparin surrogate.

In a second aspect, the disclosure provides a method for preparing a reagent as defined in the first aspect, said method comprising: (i) contacting a solid support with a solution comprising a molecule capable of binding anti-PF4 antibodies in the presence of a crosslinking agent and, optionally, a catalyser. Alternatively, the method for preparing a reagent as defined in the first aspect may comprise contacting a solid support with a solution comprising a crosslinking agent and, optionally, a catalyser, and (ii) subsequently adding a molecule capable of binding human anti-PF4 antibodies.

As outlined above, it is important for the purpose of the reagent that the molecule capable of binding anti-PF4 antibodies, for example, PF4 protein, is not itself forming a complex with heparin or a heparin surrogate before being covalently attached to the support.

A third aspect of the disclosure refers to a reagent obtainable by a method as defined in the second aspect.

A fourth aspect of the disclosure provides a composition comprising the reagent as defined in the first or third aspects of the disclosure.

A fifth aspect refers to the use of a reagent as disclosed in the first or third aspect, or a composition as defined in the fourth aspect, for detecting anti-PF4 antibodies.

A sixth aspect refers to use of a reagent as disclosed in the first or third aspect, or a composition as defined in the fourth aspect, for differentially detecting anti-PF4 antibodies (does not detect anti-PF4/heparin antibodies) and/or for diagnosing a thrombotic event not induced by heparin, for example, VITT.

A seventh aspect refers to an in vitro method for detecting human anti-PF4 antibodies comprising: (i) contacting a reagent as defined in the first or third aspects, or a composition as defined in the fourth aspect, with a biological sample obtained from a subject, and (ii) analysing the sample to detect an immunological complex formed by the reagent and the anti-PF4 antibodies, wherein formation of the immunological complex is indicative of the presence of anti-PF4 antibodies in the sample.

Since anti-PF4 antibodies are typically found in thrombotic events not induced by heparin, detection of anti-PF4 antibodies allows for diagnosing these conditions. An eight aspect thus refers to an in vitro method for diagnosing a thrombotic event not induced by heparin comprising: (i) contacting a reagent as defined in the first or third aspects, or a composition as defined in the fourth aspect, with a biological sample obtained from a subject, and (ii) analysing the sample to detect an immunological complex formed by the reagent and the anti-PF4 antibodies, wherein formation of the immunological complex is indicative of a thrombotic event not induced by heparin. Said thrombotic events not induced by heparin include VITT, as well as other thrombotic events which are not induced by heparin but also not induced by vaccination, such as, for example, spontaneous HIT-like syndrome. The presence of anti-PF4 antibodies in such thrombotic events not induced by heparin or vaccination has been confirmed, for example, in patients suffering from spontaneous HIT-like syndrome, as shown in FIG. 7 of the present disclosure. The presence of anti-PF4 antibodies is also observed in patients with severe or recurrent episodes of thrombosis and/or thrombocytopenia independent of heparin or vaccination.

The method disclosed herein overcomes many of the drawbacks of current methods for diagnosing thrombotic events not induced by heparin. First, the method has a good sensitivity. As observed from the examples below, all samples from VITT patients were correctly identified as positive when tested by the method of the disclosure, while all control samples (from healthy patients) gave a negative result (FIG. 2). The Relative Light Unit (RLU) signal values observed for said VITT samples were very high, which is indicative of a high sensitivity. Importantly, the method disclosed herein is also specific for detecting thrombotic events not induced by heparin (FIG. 6) and has no cross-reactivity with HIT (FIG. 2). This is also evidenced by the examples below, where it may be observed that all samples obtained from HIT patients tested negative with the present method. As a result, the method disclosed herein allows for differential diagnosis of VITT or other non-heparin induced thrombotic events from HIT. The method is thus a very valuable tool for the clinician, who may choose the most appropriate treatment regimens accordingly. For example, when the patient's sample is positive with the method disclosed herein, this is indicative of VITT, or other thrombotic events not induced by heparin which can be very severe. This information is valuable to the clinician, who may recommend an adjunctive measures to deescalate the FcγRIIa-dependent cell activation that triggers massive hypercoagulability. Typically, this will be achieved by administering intravenous immunoglobulin (IVIG). Additionally, when the patient's sample is positive with the method disclosed herein but negative with very specific methods for detecting anti-PF4/heparin antibodies, e.g. HemosIL® Acustar HIT-IgG_{(PF4-H)} assay and HemosIL® HIT-Ab_{(PF4-H)}, the thrombotic event is very likely not induced by heparin and, therefore, there is no need for the clinician to discontinue heparin treatment. On the contrary, when the patient's sample is negative with the method disclosed herein but positive with very specific methods for detecting anti-PF4/heparin antibodies, e.g. HemosIL® Acustar HIT-IgG_{(PF4-H)} assay and HemosIL® HIT-Ab_{(PF4-H)}, the thrombotic event is very likely induced by heparin and, therefore, the clinician should definitively consider discontinuing heparin treatment and possibly substituting it by another anticoagulant agent. Moreover, when the patient's sample is positive with the method disclosed herein and also positive for anti-PF4/heparin antibodies, the clinician should consider discontinuing heparin treatment, possibly substituting it by another anticoagulant agent, together with adjunctive measures to deescalate the FcγRIIa-dependent cell activation that triggers massive hypercoagulability, such as IVIG administration.

A ninth aspect thus refers to a method for recommending or initiating a medical regimen of a subject suspicious of suffering a thrombotic event, said method comprising detecting human anti-PF4 antibodies in a biological sample obtained from the subject by the method defined in the seventh aspect and recommending or initiating an appropriate medical regimen if human anti-PF4 antibodies are detected. As mentioned above, the appropriate medical regimen comprises measures to deescalate the FcγRIIa-dependent cell activation that triggers massive hypercoagulability, such as IVIG administration and, depending on whether anti-PF4/heparin antibodies are also present, it may also include heparin.

A tenth aspect of the disclosure provides a kit comprising a reagent as defined in the first or third aspects. An eleventh aspect provides a cartridge comprising a reagent as defined in the first or third aspects. Finally, a twelfth aspect provides for use of said kit or cartridge for detecting anti-PF4 antibodies not induced by heparin or for diagnosing thrombotic events not induced by heparin.

### BRIEF DESCRIPTION OF DRAWINGS

FIG. 1 is a schematic representation of a reagent according to the disclosure. The scheme shows a carboxyl-modified particle covalently coupled to PF4 by an amide bond.

FIG. 2 is a scatter plot showing the results of the chemiluminescence assay using carboxylated particles with covalently attached PF4 (carboxy PF4-particles, VITT P3) with three different groups of plasma samples: healthy donors (VITT−; n=39); samples from HIT patients (HIT+, n=24) and samples from patients with VITT (VITT+; n=69).

FIG. 3 is a scatter plot comparing results of the HemosIL® Acustar HIT IgG_{(PF4/H)} (HemosIL® HIT) and the carboxy PF4-particles (VITT P3) when testing plasma/serum samples from patients with suspected VITT (grey circles) and healthy donor individuals (white circles).

FIG. 4 is a scatter plot comparing results of the chemiluminescence assay using the carboxy PF4-particles (VITT P3, covalent binding of PF4), magnetic particles with carboxyl functional groups on their surface to which PF4 was adsorbed (VITT P1 and VITT P2), and HemosIL® Acustar HIT IgG_{(PF4/H)} (HemosIL) for plasma samples from patients with suspected VITT (VITT+, grey circles) and healthy donors (white circles).

FIG. 5 is a scatter plot comparing results of the chemiluminescent analysis using PF4 adsorbed onto the particle (P1) and carboxy-PF4 particles (P3, covalent coupling) for plasma samples from VITT+patients and VITT-patients.

FIG. 6 is a scatter plot comparing results of the HemosIL® Acustar HIT IgG_{(PF4/H)} (HemosIL HIT) and the carboxy PF4-particles (VITT P3) when testing two plasma samples from patients with unclear thrombotic complications (HIT and VITT negative). Sample 1 (grey circles) was analyzed at three different testing days.

FIG. 7 is a scatter plot showing the results of six patients with VITT-like pattern (thrombocytopenia, thrombosis, strongly positive anti-PF4/heparin IgG EIA and PIPA), but without previous exposure to heparin or vaccination in the HemosIL® AcuStar HIT-IgG_{(PF4-H)} assay (x-axis; cutoff: vertical line) and the new prototype VITT P3 assay (prototype VITT-IgG (PF4)) (y-axis, cutoff: horizontal line).

FIG. 8 is a scatter plot showing the results of patients with autoimmune HIT (N=70) in the HemosIL® AcuStar HIT-IgG_{(PF4-H)} assay (x-axis; cutoff: dashed vertical line) and the new prototype VITT P3 assay (prototype VITT-IgG(PF4)) (y-axis, cutoff: dashed horizontal line). 63% of the samples show positive results for the new prototype VITT-IgG(PF4) assay.

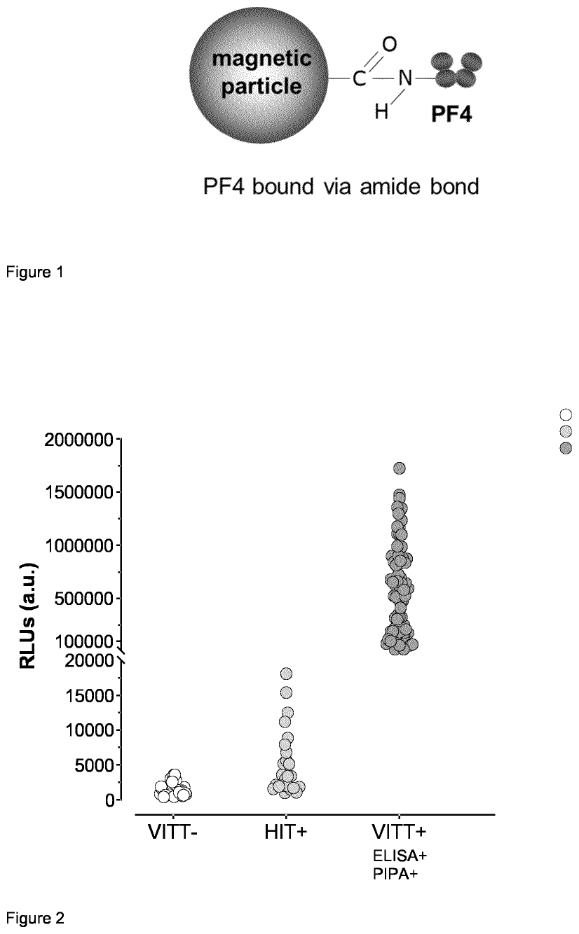

### DETAILED DESCRIPTION

All terms as used in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

The disclosure refers, in a first aspect, to a reagent comprising: (a) a binding molecule for binding anti-PF4 antibodies and (b) a solid support, wherein the binding molecule is covalently bound to the surface of the solid support.

Platelet factor 4 protein (PF4) is a small cytokine belonging to the CXC chemokine family that is also known as chemokine (C-X-C motif) ligand 4 (CXCL4). This chemokine is released from alpha-granules of activated platelets during platelet aggregation and promotes blood coagulation by moderating the effects of heparin-like molecules. In one embodiment, the mature human PF4 protein has the following sequence:

(SEQ ID NO: 1)
EAEEDGDLQCLCVKTTSQVRPRHITSLEVIKAGPHCPTAQLIATLKNGR
KICLDLQAPLYKKIIKKLLES.

In the sense of the present disclosure “anti-PF4 antibodies” are immunoglobulin G (IgG) antibodies that bind to epitopes on platelet factor 4 protein independent of heparin. “Anti-PF4/heparin or anti-PF4/H are antibodies (IgG, IgA and IgM) which recognize platelet factor 4 (PF4) associated with heparin or a heparin surrogate. The terms “anti-PF4 antibodies” (or simply “anti-PF4”) and “anti-PF4 heparin-independent antibodies” are herein used interchangeably. The terms “anti-PF4/heparin or anti-PF4/H antibodies” and “anti-PF4 heparin-dependent antibodies” are herein used interchangeably. HIT and VITT antibodies bind to different epitopes on PF4. Anti-PF4/H are found in classic HIT and require concomitant presence of PF4 and pharmacological concentrations of a relevant polyanion to effect pathogenicity. Antibodies which bind to PF4 alone (heparin-independent antibodies) are found in VITT and in other thrombotic events which are not caused either by heparin or vaccination. Both pathological classes of IgG antibodies activate platelets via the FcγRIIa. The present method allows for recognizing patients who develop thrombocytopenia and thrombosis in association with anti-PF4 antibodies heparin-independent, i.e., even in the absence of any proximate heparin exposure whatsoever. As mentioned above, some of these thrombotic events are not only found in the absence of heparin, but also in the absence of vaccination, indicating that triggers besides pharmacological heparin and Covid-19 vaccines can initiate a prothrombotic anti-PF4 disorder.

A “molecule for binding anti-PF4 antibodies”, also herein referred to simply as “binding molecule”, is generally understood as a molecule, usually, but not only, a polypeptide, that can bind anti-PF4 antibodies in a similar way to PF4 protein. To that effect, the binding molecule according to the present disclosure comprises the relevant epitope(s) of PF4. Said epitope(s) may be linear, with a consecutive stretch of amino acids, or conformational, consisting of amino acids that are distantly separated in the protein sequence and brought into proximity by the folding of the protein. These conformational epitopes may contain consecutive amino acids and non-consecutive amino acids spread throughout the protein.

The molecule can be the full-length PF4 protein, preferably human PF4 protein or a highly similar protein. In one embodiment, the binding molecule is mammalian PF4. In another embodiment, the binding molecule is primate PF4. In a particular embodiment the binding protein is human PF4. In one embodiment, the PF4 is mature PF4. The PF4 protein may be natural or synthetic. In another embodiment, the PF4 is natural, isolated PF4. In another embodiment, the PF4 is recombinant PF4. In another embodiment, the binding molecule is a polypeptide having at least 80%, at least 85%, at least 90% at least 95%, at least 97%, at least 98%, or at least 99% identity to human PF4 (SEQ ID NO: 1). In a very particular embodiment, the binding molecule is human PF4 of SEQ ID NO: 1.

In the present disclosure the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The percentage of identity determines the number of identical residues over a defined length in a given alignment. Thus, the level of identity between two sequences or (“percent sequence identity”) may be measured as a ratio of the number of identical positions shared by the sequences with respect to the number of positions compared [i.e., percent sequence identity=(number of identical positions/total number of positions compared)×100]. A gap, i.e., a position in an alignment where a residue is present in one sequence but not in the other, is regarded as a position with non-identical residues and is counted as a compared position.

A number of mathematical algorithms for rapidly obtaining the optimal alignment and calculating identity between two or more sequences are known and incorporated into a number of available software programs. For purposes of the present disclosure, the sequence identity between two amino acid sequences is preferably determined using algorithms based on global alignment, such as the Needleman-Wunsch algorithm (Needleman and Wunsch, 1970, J. Mol. Biol. 48:443-453), preferably implemented in the Needle program of the EMBOSS package (EMBOSS: The European Molecular Biology Open Software Suite, Rice et al., 2000, Trends Genet. 16:276-277); or the BLAST Global Alignment tool (Altschul et al., “Basic local alignment search tool”, 1990, J. Mol. Biol, v. 215, pages 403-410), using default settings. Local alignment also can be used when the sequences being compared are substantially the same length.

In one embodiment, the binding molecule is a polypeptide which is capable of binding anti-PF4 antibodies, for example, PF4-reactive antibodies in patients with a thrombotic event not triggered by heparin, e.g., VITT. For example, said polypeptide may be a fragment of PF4 protein, so long it maintains the ability to bind anti-PF4 antibodies. A conformational epitope within the PF4 protein has been described to bind anti-PF4 antibodies (Huynh, A. et al). Said conformational epitope is defined by the residues R22, H23, E28, K46, N47, K50, K62, and K66 of SEQ ID NO: 1. In one embodiment, the binding molecule is a polypeptide comprising the conformational epitope defined by the residues R22, H23, E28, K46, N47, K50, K62, and K66 of SEQ ID NO: 1. Said conformational epitope may also be defined as-RH-E-KN-K-K-K-. In another embodiment, the binding molecule is a fragment of PF4 which can bind anti-PF4 antibodies (from now also termed “antibody-binding PF4 fragment”). Assays for binging anti-PF4 antibodies are known in the art and may be employed by the skilled person to determine which PF4 fragments or, in general, which binding molecules, are capable of binding anti-PF4 antibodies.

In another embodiment, the binding molecule is anti-idiotype antibody of anti-PF4 antibodies. Anti-idiotype antibodies recognize an antigen-combining site of an antibody and mimic the structure and/or function of its nominal antigens. Methods to obtain anti-idiotype antibodies for known antibodies are well known to the skilled person. In another embodiment the binding molecule is an aptamer.

In particular embodiments the reagent according to the first aspect of the disclosure consists essentially of (a) a molecule capable of binding anti-PF4 antibodies, and (b) a solid support, wherein the binding molecule is covalently bound to the surface of the solid support. In particular, the binding molecule is selected from PF4, an antibody-binding fragment thereof, and an anti-idiotype antibody of anti-PF4 antibodies. The binding molecule does not contain heparin or any other heparin surrogate (i.e., is not forming a complex with heparin or another heparin surrogate). In other particular embodiments, the reagent according to the first aspect of the disclosure consists essentially of (a) PF4 and (b) a solid support, wherein PF4 is covalently bound to the surface of the solid support. Again, the PF4 in (a) is not complexed with heparin or any heparin surrogate, i.e., the molecule that is recognized and bound by the anti-PF4 antibodies does not include heparin or a surrogate thereof.

A “heparin surrogate” is understood by the skilled person as any anionic species that binds to the same heparin-binding site within the PF4 protein. An example of a heparin surrogate is polyvinyl sulfonate (PVS).

In some embodiments, the solid support of the reagent of the first aspect of the disclosure is a particle. “Particle” it is understood, as generally in the art, as a small, localized object to which can be ascribed several physical or chemical properties, such as shape, volume, density, or mass. They vary greatly in size, shape, or quantity, from subatomic particles like the electron, to microscopic particles like atoms and molecules, to macroscopic particles like powders and other granular materials. Anything that is composed of particles may be referred to as being particulate. Thus, it can be considered that the solid support in the reagent of the disclosure may be a particulate solid support.

In some embodiments, the solid support is a nanoparticle or a microparticle. Both nanoparticles and microparticles may be of different composition, such as but not limited to metal, organic, metalorganic, polymeric, quantum dots, or carbon structures. The nanoparticles and microparticles have at least two dimensions in the nanoscale, preferably all three dimensions in the nanoscale, and may have different shapes and sizes, for example spheres, rods, discs, tubes and hemispheres. In some embodiments the particulate solid support has the shape of a sphere or a bead, a rod or a tube.

In some embodiments, the particulate solid support comprises or consists essentially of a metal or metal alloy. In particular embodiments, the particulate solid support comprises or consists essentially of a magnetic metal or metal alloy. As the magnetic material, anyone that is widely used in the art may be used. For example, one of Fe₂O₃, Fe₃O₄ or FePt may be used, although not being limited thereto. Non-limiting metals appropriate for the solid support according to the disclosure may be selected from gold, platinum, silver, titanium, zinc, cerium, iron, copper, thallium, and combinations and/or alloys thereof. In other embodiments the particulate solid support comprises or consists essentially of silica. In other embodiments, the particulate solid support comprises or consists essentially of a metal-organic framework. In other embodiments, the particulate solid support is a quantum dot. In other embodiments, the particulate solid support comprises or consists essentially of carbon, for example, graphite.

In some embodiments, the solid support comprises or consists essentially of a polymer. For example, polymer based nanoparticles are known in the art and may be used in the reagent of the disclosure. Appropriate polymers for the solid support in the sense of the present disclosure may be selected from latex, polystyrene, alginic acid, gelatin, polylactic acid, chitosan, polylactide-co-glycolide, and polycaprolactone. In particular embodiments, the solid support comprises or consists essentially of latex or polystyrene.

The binding molecule, for example, PF4, may be covalently bound to the solid support, for example, via an amide bond, an amine bond or a thioether bond. In particular embodiments of the first aspect of the disclosure, the binding molecule, for example, PF4, is bound to the solid support via an amide bond. In some embodiments, the reagent of the first aspect may further comprise a spacer. Said “spacer” may be any branched or unbranched carbon chain comprising at least one carbon, which is bound to the solid support on the one side and to the binding moiety on the other side. Preferably, the binding molecule, for example, PF4, is bound to the spacer via an amide bond. In particular embodiments, the reagent of the disclosure may be seen to have the following structure:

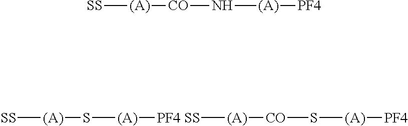

wherein SS is a solid support, A is a spacer which can be present or absent from the reagent, and PF4 is a binding molecule capable of binding anti-PF4 antibodies.

The nature of the spacer according to the disclosure is not limited so long as it does not negatively affect the function of the reagent, in particular with respect to the binding of anti-PF4 antibodies. Non-limiting spacers for the reagent of the disclosure are amino acids, peptides, polyethylene glycol, alkyl groups, etc. In a particular embodiment the reagent of the first aspect of the disclosure can have the structure: SS-CO-NR-PF4, wherein R is H or (C1-C4) alkyl. More particularly, the reagent can have the structure SS-CO-NH-PF4

Coupling of polypeptides and proteins to a solid support, including to different kinds of particles, which in turn include beads, may be done by several methods which are well known to the skilled person. An overview of some of the main methods is given by Di Marco et al (Int J Nanomedicine. 2010; 5:37-49) and Biju (Chem. Soc. Rev., 2014,43, 744-764), which is hereby included by reference. Some methods comprise chemically coupling the polypeptides to the solid support using established reagents such bifunctional cross-linker molecules. These methods may be used for preparing the reagent of the present disclosure.

A second aspect refers to a method for preparing a reagent as defined above generally comprising (i) contacting a solid support with a solution comprising a crosslinking agent and, optionally, a catalyser, and (ii) adding a molecule capable of binding anti-PF4 antibodies. Steps (i) and (ii) may take place one after the other (step ii after step i) or simultaneously, i.e., the solid support is contacted with a solution comprising both the crosslinking agent and the binding molecule, and optionally, the catalyser. All embodiments described above for the binding molecule and the solid support also apply to the method of the second aspect of the disclosure.

The solid support may contain functional groups which may react with the crosslinking agent. In some embodiments, the solid support contains functional groups selected from carboxyl, hydroxyl, sulfhydryl, tosyl, and amino functional groups. In some embodiments, the solid support is decorated with negatively charged functional groups. In a particular embodiment, the solid support is carboxylated.

Many crosslinkers are available in the market and they can be chosen for specific needs (such as chemical specificity, spacer arm length, cleavability). Non-limiting crosslinkers for use in the present disclosure based on their respective functions are shown in Table 1.

TABLE 1
Cross-linkers for covalently coupling polypeptides and proteins
to a particle based on their respective functions

| | | Functional | Functional |
|---|---|---|---|
| | E.g. of | groups on | groups on |
| | functional | nanoparticles/ | proteins/ |
| Reactive groups | cross-linker | proteins | nanoparticles |
| —NHS ester | SIAB, SMCC, | —NH₂ | —SH |
| Maleimide or | SPDP, SPMB, | | |
| lodoacetamides | MBS | | |
| Carbodiimide | EDC or EDAC, | —COOH | —NH₂ |
| | DCC (+NHS | | |
| | or sulfo-NHS | | |
| | catalyzer) | | |
| —NHS ester | EGS, DSP, | —NH₂ | —NH₂ |
| | DSS, BS3 | | |
| Maleimide | BMME | —SH | —SH |

Abbreviations: SIAB, N-succinimidyl(4-iodoacetyl) aminobenzoate; SMCC, succinimidyl-4-(N-maleimidomethyl)cyclohexana-1-carboxylate; MBS, m-maleimidobenzoyl-N-hydroxysuccimide ester; SPDP, succinimidyl 3-(2-pyridyldithio) propionate; SPMB, succinimidyl (4-p-maleimidophenyl) butyrate; EDC, 1-Ethyl-3-[3-dimethylaminopropyl] carbodiimide hydrochloride; DCC, dicyclohexyl carbodiimide; NHS, N-hydroxysuccinimide; EGS, ethylene glycolbis(succinimidylsuccinate); BS3, bis-(sulfosuccinimidyl) suberate; BMME, bis(maleimido methyl) ether; DSS, disuccinimidyl suberate; DSP, dithiobis succinimidyl propionate); -NHS, N-Hydroxysuccinimide.

In a particular embodiment, the crosslinking agent is a carbodiimide. In a particular embodiment, the method of the disclosure takes place in the presence of a catalyzer, for example NHS.

In other embodiments the binding molecule can be immobilized using molecular or genetic engineering techniques, such as intein-mediated protein ligation, click chemistry, or other similar technologies.

The method of the second aspect takes place under conditions to promote covalent binding of the binding molecule to the solid support. Appropriate conditions may be established by the skilled person and may include, in one embodiment, incubating the solid support with a solution comprising the crosslinker, optionally, the catalyser, and the binding molecule for a sufficient amount of time, for example from 20 minutes to 5 hours. In a particular embodiment the incubation time is 1-4 hours. When the method of the disclosure takes place in two separate steps, one embodiment contemplates for step (i) an incubation time of at least 15 min, for example from 15 min to 1 hour, and for step (ii) an incubation time of 1 to 5 hours. In a particular embodiment, step (i) contemplates incubating from 20 min to 45 min and step (ii) contemplates incubating from 1 to 3 h. In particular embodiments, the incubations take place at room temperature. In particular embodiment the incubations take place under continuous mixing. In some embodiments the method of the disclosure takes place in the presence of a buffer. In particular embodiments, steps (i) and (ii) take place at a slightly acidic pH, for example at a pH ranging from 5 to 6.5.

In some embodiments, the method further comprises a step, subsequent to (ii), of adding a blocking agent (step (iii). The blocking agent blocks the PF4-free areas of the solid support to avoid un-specific binding to the reagent. Non-limiting blocking agents are albumin, polyethylene glycol and casein. In a particular embodiment, the blocking agent is albumin.

The method may also comprise another step, prior to (i), which comprises activating the solid support. Activation may proceed by washing with a mildly acidic buffer, for example, a pH ranging from 5 to 6.

As mentioned above, in most embodiments the solid support contains functional groups. Supports containing active functional groups are commercially available. For example, a wide variety of functionalized particles of various sizes and forms are readily available in the market. If the solid support does not contain functional groups, then a first step of the method of the disclosure may comprise functionalizing the solid support. The skilled person can perform this step following standard procedures, which will depend on the functional groups desired.

A third aspect of the disclosure refers to a reagent obtained by the above method. Said reagent may bind anti-PF4 antibodies.

The disclosure also provides a composition comprising the reagent of the disclosure. In some embodiments, the composition comprises further components, which can be selected from a buffer, a stabilizing compound, a preservative (e.g., an antimicrobial agent), and combinations thereof.

Further aspects of the disclosure refer to the use of a reagent of the first or third aspects of the disclosure for detecting anti-PF4 antibodies or for diagnosing thrombotic events not induced by heparin, such as vaccine induced thrombotic thrombocytopenia (VITT) and other thrombotic events neither induced by heparin or vaccination. “Thrombotic event not induced by heparin” is understood as a thrombotic event resulting from the activation of platelets and other blood cells or the endothelium by anti-PF4 antibodies which arise against PF4 alone or unknown triggers. other than heparin. Non-limiting examples of thrombotic events not triggered by heparin are VITT, spontaneous HIT-like syndrome and other unclear thrombosis. The term “spontaneous HIT-like” has been used to refer to the condition by which patients develop thrombocytopenia and thrombosis in association with platelet-activating anti-PF4 antibodies even in the absence of proximate heparin exposure. It is apparent that these patients' sera are able to activate platelets in the absence of heparin, i.e., the serum contains anti-PF4 heparin-independent antibodies.

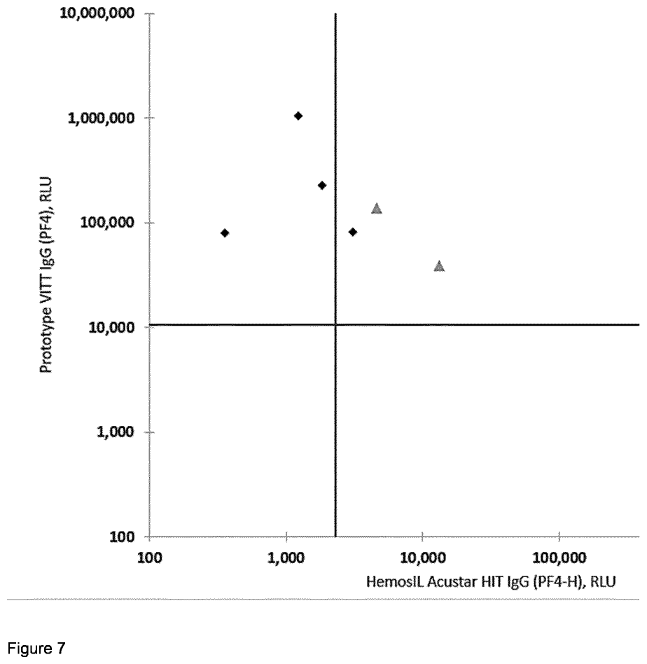

The present disclosure shows that some patients with spontaneous HIT-like syndrome have anti-PF4 heparin-dependent (anti-PF4/H) and anti-PF4 heparin-independent antibodies (anti-PF4), although some patients feature anti-PF4 heparin-independent antibodies (anti-PF4) alone (see FIG. 7). In particular embodiments, the reagent of the first or third aspects of the disclosure is for diagnosing VITT. In particular embodiments, the reagent of the first or third aspects of the disclosure is for diagnosing VITT caused by COVID-19 vaccination. In another embodiment, reagent of the first or third aspects of the disclosure is for diagnosing a thrombotic event that is not induced by heparin or by vaccination, such as, for example, spontaneous HIT-like syndrome. In particular embodiments, the reagent of the first or third aspects of the disclosure is for diagnosing unclear thrombosis. All embodiments described above for the reagents of the first and third aspects of the disclosure also apply to their use.

In patients who present with thrombocytopenia and catastrophic venous and arterial thrombosis, the presence of anti-PF4 heparin-dependent and heparin-independent antibodies should be considered as potential causes, even in the absence of proximate heparin exposure. This is highly relevant as these patients need adjunctive treatment, e.g. with IVIG. For the first time this is now made possible by the present method.

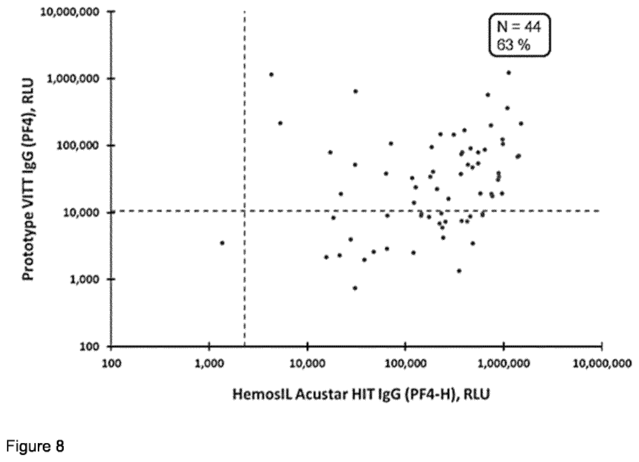

It is further noted that severe, atypical presentations of HIT (“autoimmune HIT”) are associated with both HIT-like (heparin-dependent) and VITT-like (heparin-independent) antibodies, as is shown in FIG. 8. Again, a key concept here is that the presence of anti-PF4 heparin-independent antibodies require anticoagulation plus an adjunct treatment, namely high-dose IVIG, to deescalate the severe anti-PF4 IgG-mediated hypercoagulability state. In one embodiment, the reagent of the first or third aspects of the disclosure may be used for diagnosing atypical HIT.

The seventh and eight aspects of the disclosure refer to in vitro methods for detecting anti-PF4 antibodies and for diagnosing a thrombotic event not induced by heparin, respectively. The methods comprise (i) contacting a reagent as defined in the first and third aspects or a composition comprising said reagent with a biological sample obtained from a subject and (ii) analysing the sample to determine if a complex between the reagent and anti-PF4 antibodies has been formed, wherein formation of the complex is indicative of the sample containing anti-PF4 antibodies not induced by heparin, which, in turn, is indicative of a thrombotic event not induced by heparin. Non-limiting examples of thrombotic events not triggered by heparin are VITT, spontaneous HIT-like syndrome and other unclear thrombosis. In particular embodiments, the method of the disclosure is for diagnosing VITT. In particular embodiments, the method of the disclosure is for diagnosing VITT caused by COVID-19 vaccination. In particular embodiments, the method of the disclosure is for diagnosing a thrombotic event that is not induced by heparin or by vaccination, such as, for example, spontaneous HIT-like syndrome. In particular embodiments, the method of the disclosure is for diagnosing thrombotic complications of unclear origin. In particular embodiments, the method of the disclosure is for diagnosing atypical HIT. All these conditions can be diagnosed by the present method because they present with anti-PF4 antibodies. All embodiments described above for the reagents of the first and third aspects of the disclosure also apply to these methods.

The biological sample is preferably whole blood or part of whole blood, e.g., plasma or serum. In a particular embodiment the biological sample is citrated plasma or serum. In other embodiments the biological sample is plasma anticoagulated by calcium chelators such as EDTA or anti-thrombin reagents such as hirudin or PPACK, or anticoagulatory aptamers.

The diagnosis method of the disclosure is based in the reagent of the disclosure specifically binding to anti-PF4 antibodies, when said antibodies are present in the sample. The reagent comprises a binding molecule (a) which may recognise and capture the anti-PF4 antibodies. As shown in the examples below, the reagent of the disclosure does not detect anti-PF4/H (anti-PF4 antibodies induced by heparin) (FIG. 2). This allows for differential diagnosis of HIT and non-heparin induced thrombotic events.

In one embodiment, the method is for the differential diagnosis of a thrombotic event not induced by heparin. For example, the method is for differentiating between HIT and a thrombotic event not induced by heparin. In a particular embodiment, the method is for differentiating between HIT and VITT, in particular between HIT and VITT caused by COVID-19 vaccination.

A ninth aspect refers to a method for recommending or initiating a medical regimen of a subject suspicious of suffering a thrombotic event, said method comprising detecting human anti-PF4 antibodies in a biological sample obtained from the subject by the method defined above and recommending or initiating an appropriate medical regimen if human anti-PF4 antibodies are detected. As mentioned above, the possibility of detecting the presence of anti-PF4 antibodies (heparin-independent) is of great value to the clinician, who may choose the most appropriate treatment regimen accordingly. When the patient's sample is positive with the method disclosed herein (indicating presence of anti-PF4 antibodies), this is indicative of VITT, or other thrombotic events not induced by heparin, which can be very severe. This information is valuable to the clinician, who may recommend an adjunctive measures to deescalate the FcγRIIa-dependent cell activation that triggers massive hypercoagulability. Typically, this will be achieved by administering intravenous immunoglobulin (IVIG). Additionally, when the patient's sample is positive with the method disclosed herein but negative with very specific methods for detecting anti-PF4/heparin antibodies, e.g. HemosIL® Acustar HIT-IgG_{(PF4-H)} assay and HemosIL® HIT-Ab_{(PF4-H)}, the thrombotic event is very likely not induced by heparin and, therefore, there is no need for the clinician to discontinue heparin treatment. On the contrary, when the patient sample is negative with the method disclosed herein but positive with very specific methods for detecting anti-PF4/heparin antibodies, e.g. HemosIL® Acustar HIT-IgG_{(PF4-H)} assay and HemosIL® HIT-Ab_{(PF4-H)}, the thrombotic event is very likely induced by heparin and, therefore, the clinician should definitively consider discontinuing heparin treatment (possibly substituting it by another anticoagulant agent), while possibly recommending IVIG administration (or an equivalent treatment). Moreover, when the patient sample is positive with the method disclosed herein and also positive for anti-PF4/heparin antibodies, the clinician should consider discontinuing heparin treatment, possibly substituting it by another anticoagulant agent, together with adjunctive measures to deescalate the FcγRIIa-dependent cell activation that triggers massive hypercoagulability, such as IVIG administration. The disclosure also provides for a method for treating a thrombotic event in a subject in need thereof, said method comprising performing the method of the eighth aspect to determine if the patient suffers from a thrombotic event not induced by heparin and, if this is the case, administering an appropriate medical regimen, wherein the appropriate medical regimen may include heparin.

An in vitro method for the differential diagnosis of HIT and non-heparin induced thrombotic events is also provided comprising (1) performing the in vitro method of the seventh aspect of the disclosure and (2) performing an in vitro diagnosis for HIT, wherein when (1) is positive and (2) is negative, this is indicative of a thrombotic event not triggered by heparin and HIT may be disregarded. If HIT is disregarded, the medical regime to be administered to the patient may comprise heparin. When (1) is negative and (2) is positive, this is indicative of HIT and administration of heparin to the patient should be discontinued. The in vitro diagnosis for HIT may be performed, e.g., by use of such specific assays as HemosIL® Acustar HIT-IgG_{(PF4-H)} or HemosIL® HIT-Ab_{(PF4-H)}. The in vitro diagnosis for HIT may additionally or alternatively be performed by a functional assay.

As outlined above, the method of the disclosure is based in the binding of the reagent of the disclosure to anti-PF4 antibodies, when said antibodies are present in the sample, to form a detectable complex. In particular embodiments, the in vitro methods disclosed above comprise: (i) contacting a reagent as defined in the first or third aspects, or a composition as defined in the fourth aspect, with a biological sample obtained from a subject to form a immunological complex between the reagent and anti-PF4 antibodies, if present, and (ii) analysing the sample to determine if the immunological complex has been formed, wherein formation of the complex is indicative of the sample containing anti-PF4 antibodies.

The immunological complex formed by the reagent and the anti-PF4 antibodies may be directly detectable, for example, when the reagent emits a detectable signal which varies when it forms an immunological complex with the anti-PF4 antibodies. This may be achieved by selecting certain solid supports. Solid supports emitting a detectable signal are known to the skilled person and may be used for preparing the reagent of the disclosure. Non-limiting examples of said solid supports are gold nanoparticles and quantum dots. In another example, the immunological complex formed by the reagent and the anti-PF4 antibodies may be directly detectable in a turbidimetric assay. This may be achieved by using latex particles as solid support. It may also be achieved by using polystyrene particles as solid support.

In another embodiment, the in vitro methods disclosed above further comprise contacting the mixture obtained from (i) with a tracer capable of binding to the captured anti-PF4 antibodies. In a particular embodiment, the tracer comprises a label. In another particular embodiment, the label is a detectable molecule selected from the group consisting of a chemiluminescent molecule, a chromogenic molecule, a fluorescent molecule, a radioactive molecule, a quantum dot, colloidal gold and a gold nanoparticle. In some embodiments the label is a molecule that becomes detectable upon contact with a trigger. In another embodiment, the tracer comprises an enzyme that may trigger a detectable change in a substrate present in the sample. In another embodiment the tracer comprises a substrate that may undergo a detectable change, optionally, in the presence of an appropriate trigger. In a particular embodiment, the tracer is a labelled anti-human IgG antibody. In a very particular embodiment, the tracer is an anti-human IgG antibody labelled with a chemiluminescent molecule. Chemiluminescent molecules are well known in the art and may include luminol, isoluminol, N-(4-aminobutyl)-N-ethylisoluminol (ABEI), acridinium, etc. In a particular embodiment, the in vitro methods for detecting anti-PF4 antibodies or thrombotic events not induced by heparin further comprise contacting the mixture obtained from (i) with a tracer capable of binding to the captured anti-PF4 antibodies and a trigger that induces the tracer to undergo a detectable change.

The tenth aspect of the disclosure provides a kit for performing any of the in vitro methods disclosed above (e.g. detecting anti-PF4 antibodies, or diagnosing thrombotic event not induced by heparin, or recommending an appropriate medical regime for a patient suffering or suspected of suffering from a thrombotic event). In a particular embodiment the kit comprises, in separate containers, (i) a reagent as defined in the first or third aspects of the disclosure, or a composition comprising the same as defined in the fourth aspect, and (ii) a tracer capable of binding to the anti-PF4 antibodies. In another embodiment the kit may also comprise a trigger solution for inducing a detectable change in the tracer. In further embodiments the kit may additionally comprise buffer solutions, diluents, stabilizers, preservatives and/or instructions for use in detecting anti-PF4 antibodies or diagnosing thrombotic events not induced by heparin.

The disclosure also provides a cartridge for detecting anti-PF4 antibodies or diagnosing thrombotic events, or recommending appropriate medical regime, all as disclosed above.

In a particular embodiment the cartridge comprises, in separate containers, (i) a reagent as defined in the first or third aspects of the disclosure, or a composition comprising the same as defined in the fourth aspect, and (ii) a tracer capable of binding to the anti-PF4 antibodies. In another embodiment the cartridge may also comprise a trigger solution for inducing a detectable change in the tracer. In further embodiments the cartridge may additionally comprise buffer solutions, diluents, stabilizers, preservatives and/or instructions for use in any of the methods described above.

In particular embodiments the reagent in the kit or cartridge comprises particles as solid support, for example, magnetic particles. In another embodiment, the cartridge allows for automated testing of the samples to detect anti-PF4 antibodies or diagnose a thrombotic event as defined above, or to recommend an appropriate treatment to a patient as defined above. The cartridge may contain all components required for the testing, for example, the cartridge may contain the reagent in the form of a particle suspension, assay buffer, tracer and diluent. The components in the cartridge may be released from their containers and mixed with the sample allowing for automated testing, which is a relevant advantage of the method of the disclosure. In a particular embodiment the cartridge is for use in automated testing with an automated instrument. The automated instrument fits the cartridge and automatically detects the signal emitted by the tracer when anti-PF4 antibodies are present in the sample. In a particular embodiment the automated instrument is ACL AcuStar (Werfen).

For completeness, the present disclosure is further defined in the following numbered embodiments:
1. A reagent comprising:
   (a) a molecule for binding anti-PF4 antibodies (binding molecule); and
   (b) a solid support,
   wherein the binding molecule (a) is covalently bound to the surface of the solid support (b), and wherein the binding molecule (a) does not include heparin or a heparin surrogate.
2. A reagent consisting essentially of:
   (a) a molecule for binding anti-PF4 antibodies (binding molecule); and
   (b) a solid support,
   wherein the binding molecule (a) is covalently bound to the surface of the solid support (b), and wherein the binding molecule (a) does not include heparin or a heparin surrogate.
3. The reagent according to any one of the preceding embodiments, wherein the binding molecule is PF4 protein.
4. The reagent according to the preceding embodiment, wherein the binding molecule is human PF4 protein.
5. The reagent according to any one embodiments 1-2, wherein the binding molecule is a polypeptide having at least 80%, at least 85% or at least 90% identity to human PF4 (SEQ ID NO: 1).
6. The reagent according to the preceding embodiment, wherein the binding molecule is a polypeptide having at least 95%, at least 97%, at least 98%, or at least 99% identity to human PF4 (SEQ ID NO: 1).
7. The reagent according to any one embodiments 1-2, wherein the binding molecule is an antibody-binding fragment of the PF4 protein.
8. The reagent according to any one embodiments 1-2, wherein the binding molecule is an anti-idiotype antibody of anti-PF4 antibodies.
9. The reagent according to any one embodiments 1-2, wherein the binding molecule is a polypeptide comprising the conformational epitope defined by the residues R22, H23, E28, K46, N47, K50, K62, and K66 of SEQ ID NO: 1
10. The reagent according to any one of the preceding embodiments, wherein the solid support is a particle, in particular, a microparticle or nanoparticle.
11. The reagent according to any one of the preceding embodiments, wherein the solid support comprises or consists essentially of a metal.
12. The reagent according to the preceding embodiment, wherein the metal is a magnetic metal.
13. The reagent according to the preceding embodiment, wherein the metal is selected from the group consisting of Fe2O3, Fe3O4 and FePt.
14. The reagent according to any one of embodiments 1-10, wherein the solid support comprises or consists essentially of a polymer.
15. The reagent according to the preceding embodiment, wherein the polymer is selected from latex and polystyrene.
16. The reagent according to any one of the preceding embodiments, wherein the PF4 is bound to the solid support via an amide bond, an amine bond or a thioether bond.
17. The reagent according to the preceding embodiment, wherein the PF4 is bound to the solid support via an amide bond.
18. The reagent according to any one of the preceding embodiments, wherein the reagent further comprises a spacer.
19. The reagent according to any one of the preceding embodiments, wherein the reagent has the following structure:
   SS-(A)-CO-NH-(A)-PF4, or
   SS-(A)-S-(A)-PF4 SS-(A)-CO-S-(A)-PF4,
   wherein SS is the solid support, A is a spacer which can be present or absent from the reagent, and PF4 is the binding molecule capable of binding anti-PF4 antibodies.
20. The reagent according to the preceding embodiment, wherein the reagent has the following structure: SS-CO-NR-PF4, wherein R is H or (C1-C4) alkyl.
21. A method for preparing a reagent as defined in any one of the preceding claims, said method comprising:
   (i) contacting a solid support with a solution comprising a in the presence of a crosslinking agent and, optionally, a catalyser, and
   (ii) adding a molecule capable of binding anti-PF4 antibodies (binding molecule), or, alternatively, said method comprising:
   (i) contacting a solid support with a solution comprising a molecule capable of binding anti-PF4 antibodies (binding molecule) in the presence of a crosslinking agent and, optionally, a catalyser.
22. The method according to the preceding embodiment, wherein the solid support contains functional groups selected from carboxyl, hydroxyl, sulfhydryl, and amino functional groups.
23. The method according to embodiment 21, wherein the solid support contains negatively charged functional groups.
24. The method according to the preceding embodiment, wherein the solid support is carboxylated.
25. The method according to any one of embodiments 21-24, wherein the crosslinking agent is a carbodiimide.
26. The method according to any one of embodiments 21-24, wherein the crosslinking agent is a N-Hydroxysuccinimide ester
27. The method according to any one of embodiments 21-24, wherein the crosslinking agent is a Maleimide.
28. The method according to any one of embodiments 21-27, wherein the binding molecule is PF4 protein.
29. The method according to the preceding embodiment, wherein the binding molecule is human PF4 protein.
30. The method according to any one embodiments 21-27, wherein the binding molecule is a polypeptide having at least 80%, at least 85% or at least 90% identity to human PF4 (SEQ ID NO: 1).
31. The method according to the preceding embodiment, wherein the binding molecule is a polypeptide having at least 95%, at least 97%, at least 98%, or at least 99% identity to human PF4 (SEQ ID NO: 1).
32. The method according to any one embodiments 21-27, wherein the binding molecule is an antibody-binding fragment of the PF4 protein.
33. The method according to any one embodiments 21-27, wherein the binding molecule is an anti-idiotype antibody of anti-PF4 antibodies.
34. The method according to any one embodiments 21-27, wherein the binding molecule is a polypeptide comprising the conformational epitope defined by the residues R22, H23, E28, K46, N47, K50, K62, and K66 of SEQ ID NO: 1
35. The method according to any one embodiments 21-34, wherein the solid support is a particle, in particular, a microparticle or nanoparticle.
36. The method according to any one of embodiments 21-35, wherein the solid support comprises or consists essentially of a metal.
37. The method according to the preceding embodiment, wherein the metal is a magnetic metal.
38. The method according to the preceding embodiment, wherein the metal is a magnetic metal is selected from the group consisting of Fe2O3, Fe3O4 and FePt.
39. The method according to any one of embodiments 21-35, wherein the solid support comprises or consists essentially of a polymer.
40. The method according to the preceding embodiment, wherein the polymer is selected from latex and polystyrene.
41. The method according to any one of embodiments 21-40, wherein the method takes place in the presence of a catalyzer.
42. The method according to the preceding embodiment, wherein the catalyser is N-Hydroxysuccinimide.
43. The method according to any one of embodiments 21-42, comprising a further step of adding a blocking agent.
44. The method according to any one of embodiments 21-43, comprising an initial step of activating the solid support.
45. A reagent obtained by a method as defined in any one of embodiments 21-44.
46. A composition comprising a reagent as defined in any one of embodiments 1-20 or 45.
47. The composition according to the preceding embodiment further comprising a component selected from the group consisting of from a buffer, a stabilizing compound, a preservative (e.g., an antimicrobial agent), and combinations thereof.
48. Use of a reagent as defined in any one of embodiments 1-20 or 45 or a composition as defined in any one of embodiments 46-47 for detecting anti-PF4 antibodies.
49. Use of a reagent as defined in any one of embodiments 1-20 or 45 or a composition as defined in any one of embodiments 46-47 for diagnosing thrombotic events not induced by heparin or for diagnosing atypical HIT.
50. An in vitro method for detecting anti-PF4 antibodies comprising:
   (i) contacting a reagent as defined in any one of embodiments 1-20 or 45 or a composition as defined in any one of embodiments 46-47 with a biological sample obtained from a subject, and
   (ii) analysing the sample to detect a complex formed by the reagent and the anti-PF4 antibodies,
   wherein detecting the complex is indicative of the sample containing anti-PF4 antibodies.
51. The in vitro method according to the preceding claim, that does not detect anti-PF4/H antibodies.
52. An in vitro method for diagnosing a thrombotic event not induced by heparin comprising:
   (i) contacting a reagent as defined in any one of embodiments 1-20 or 45 or a composition as defined in any one of embodiments 46-47 with a biological sample obtained from a subject, and
   (ii) analysing the sample to detect a complex formed by the reagent and the anti-PF4 antibodies,
   wherein detecting the complex is indicative of a thrombotic event not induced by heparin.
53. The method according to any one of embodiments 50-52, further comprising contacting the mixture obtained from (i) with a tracer, in particular, a tracer capable of binding to the anti-PF4 antibodies.
54. The method according to the preceding embodiment, wherein the tracer comprises a detectable label.
55. The method according to the preceding embodiment, wherein the detectable label is selected from the group consisting of a chemiluminescent molecule, a chromogenic molecule, a fluorescent molecule.
56. The method according to the preceding embodiment, wherein the detectable label is a chemiluminescent molecule.
57. The method according to any of the embodiments 53-56, wherein the tracer comprises a labelled anti-human IgG antibody.
58. The method according to any of the embodiments 50-57, wherein the biological sample is selected from whole blood, plasma and serum.
59. The method according to the preceding embodiment, wherein the biological sample is plasma or serum.
60. The method according to any one of embodiments 52-59, that is for the differential diagnosis of thrombotic events not induced by heparin and heparin-induced thrombotic events.
61. The method according to the preceding embodiment, wherein the heparin-induced thrombotic event is HIT.
62. The method according to embodiment 60, wherein the thrombotic event not induced by heparin is selected from VITT and a thrombotic event not induced by vaccination.
63. The method according to the preceding embodiment, wherein the thrombotic event not induced by heparin or vaccination is spontaneous HIT or unclear thrombosis.
64. The method according to any one of embodiments 60-63, further comprising performing an in vitro diagnosis for HIT.
65. The method according to the preceding embodiment, wherein the in vitro diagnosis for HIT is performed by means of an immunoassay selected from the group consisting of HemosIL® Acustar HIT-IgG_{(PF4-H)} assay and HemosIL® HIT-Ab_{(PF4-H)}.
66. The method according to embodiment 64, wherein the in vitro diagnosis for HIT is performed by means of a functional assay.
67. A kit comprising:
   (i) a reagent as defined in any one of embodiments 1-20 or 45 or a composition as defined in any one of embodiments 46-47, and
   (ii) optionally, a further component selected from a tracer capable of binding to the anti-PF4 antibodies, an assay buffer, a diluent, and combinations thereof.
68. A cartridge comprising:
   (i) a reagent as defined in any one of embodiments 1-20 or 45 or a composition as defined in any one of embodiments 46-47, and
   (ii) optionally, a further component selected from a tracer capable of binding to the anti-F4 antibodies, an assay buffer, a diluent, and combinations thereof.
69. Use of a kit as defined in embodiment 65 or a cartridge as defined in embodiment 66, for detecting anti-PF4 antibodies, or for diagnosing a thrombotic event not induced by heparin.
70. A method for recommending or initiating a medical regimen of a subject suspicious of suffering a thrombotic event, said method comprising
   (a) detecting human anti-PF4 antibodies in a biological sample obtained from the subject by a method defined in any one of embodiments 50-51, and
   (b) recommending or initiating an appropriate medical regimen if human anti-PF4 antibodies are detected.
71. The method according to any one of embodiment 70, wherein the medical regime comprises measures to deescalate the FcγRIIa-dependent cell activation that triggers massive hypercoagulability.
72. The method according to the preceding embodiment, wherein the measures comprise administering intravenous immunoglobulin (IVIG).
73. The method according to any one of embodiments 70-72, further comprising detecting anti-PF4/H antibodies in the patient's sample, wherein:
   (a) when the sample is positive for anti-PF4 antibodies and negative for anti-PF4/H antibodies, heparin may be administered, or
   (b) when the sample is positive for anti-PF4/H antibodies, heparin is discontinued.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### EXAMPLES

### 1. Magnetic Particles Preparation

1.1. Carboxyl-modified magnetic particles (Dynabeads M-270 Carboxylic Acid) are washed with 2-(N-morpholino) ethanesulfonic acid (MES) buffer pH 5.8 for ten minutes with homogeneous mixing. After removing the supernatant using a magnet, the particle pellet is resuspended in a working solution of EDAC for 30 minutes at room temperature (20-25° C.), with constant mixing. Activated particles are washed in coupling buffer (MES or PBS buffer saline) and mixed with a volume of PF4 solution. The mixture is incubated for 2-3 hours at room temperature (20-25° C.) with continuous mixing. Immediately after, the PF4-coupled particles are repositioned on a magnet to remove the supernatant and rinsed in a phosphate buffer containing BSA as a blocking molecule. After the final rinse cycle is complete, blocked particles are placed on a magnetic separator, supernatant removed by magnetic separation and particles resuspended in PBS buffer saline with BSA and stored at 2° C.˜8° C. until use. These particles are herein referred to as VITT P3, or carboxy PF4-particles, and contained PF4 covalently bound to the magnetic particles.
1.2. Preparation of magnetic particles to which PF4 was adsorbed. Protein adsorption was carried out at pH 7.5 (VITT P1) and at pH 5.8 (and VITT P2) magnetic particles. Carboxyl-modified magnetic particles (Dynabeads M-270 Carboxylic Acid) are washed with 50 mM phosphate buffer, pH 7.5 (VITT P1 particles) or 50 mM MES buffer pH 5.8 (VITT P2) for ten minutes with homogeneous mixing. After removing the supernatant using a magnet, the particle pellet is resuspended in a PF4 solution (PF4 diluted in 50 mM phosphate buffer, pH 7.5 for VITT P1; or PF4 diluted in 50 mM MES PH 5.8 for VITT P2). The mixture is incubated for 2-3 hours at room temperature (20-25° C.) with continuous mixing. Immediately after, the PF4-coupled particles are repositioned on a magnet to remove the supernatant and rinsed in a phosphate buffer containing BSA as a blocking molecule. After the final rinse cycle is complete, blocked particles are placed on a magnetic separator, supernatant removed by magnetic separation and particles resuspended in PBS buffer saline with BSA and stored at 2° C.˜8° C. until use

TABLE 1
Main features of the magnetic particles used in the examples

| | Magnetic | Molecule | Immobilization |
|---|---|---|---|
| Reagent | beads | attached | Type |
| VITT P1 | Carboxyl-activated | PF4 | Adsorption -- neutral pH |
| VITT P2 | Carboxyl-activated | PF4 | Adsorption - acidic pH |
| VITT P3 | Carboxy-activated | PF4 | Covalent |

### 2. VITT Samples

Plasma samples from patients with suspected VITT were kindly provided by Dr. Andreas Greinacher, Universitätsmedizin Greifswald, reference laboratory for VITT and HIT. Cases had been identified as suspected VITT in hospital Universitätsmedizin Greifswald on the grounds of clinical presentation, radiological evidence of thrombosis, local laboratory results for platelet count, coagulation parameters and a positive test for PF 4 dependent, platelet activating antibodies. Case definition is: presentation between 5 and 28 days post-ChAdOx1 n COV-19 vaccine; thrombosis and thrombocytopenia (platelets <150×109/L), or isolated thrombocytopenia; evidence of extreme activation of the coagulation system (D-dimers >4000 μg/L, or >2000 μg/L with a strong clinical index of suspicion). These cases are categorized into those with unlikely, possible, or probable VITT. All samples analyzed in this study were collected before treatment for VITT.

Plasma samples from patients confirmed of suffering from HIT were kindly provided by Dr. Andreas Greinacher, Universitätsmedizin Greifswald. The patients had been diagnosed as having HIT by Dr. Andreas Greinacher, Universitätsmedizin Greifswald, reference laboratory for VITT and HIT.

Plasma samples from patients with thrombotic complications of unclear origin were kindly provided Dr. Andreas Greinacher, Universitätsmedizin Greifswald, reference laboratory for VITT and HIT. The patients had been studied by Andreas Greinacher, Universitätsmedizin Greifswald, reference laboratory for VITT and HIT where HIT and VITT had been disregarded after running the appropriate tests. Plasmas from healthy donors were used as control samples.

### 3. Determination of VITT

The following assays were employed on the ACL AcuStar™ system to analyze the samples: commercial anti-PF4 ELISA (LIFECODES PF4 IgG assay), HemosIL® AcuStar™ HIT-IgG_{(PF4-H)} (Werfen) and the particles obtained in section 1 (VITT assay). Manufacturer's instructions were followed with commercial reagent kits. The following protocol was applied for the VITT assay:
1. 15 μl of anti-PF4 antibodies containing sample was 10-fold prediluted with sample diluent (135 μl) before mixing with the assay reagents.
2. 15 μl of prediluted sample was dispensed into a cuvette and mixed with 70 μl of assay buffer (AB) and 20 μl of magnetic particles (MPs). The mixture was incubated for 8.5 minutes.
3. The formed complex (particle with anti-PF4 antibodies) was separated using a magnet, and the complex was aspirated and washed with rinse solution. This washing step was repeat-ed 3 times.
4. 160 μl of tracer was dispensed into the complex and the mixture was incubated for 9.5 min. The resulting complex (particle with antibodies plus tracer) was separated with a magnet and washed four times with rinse solution.
5. 190 μl of trigger solution A and & 200 μl of trigger solution B was dispensed to initiate the chemiluminescent reaction; and
6. Relative Light Units (RLU) results were reported.

RLUs readings from the ACL AcuStar™ equipment were determined as a function of the concentration of the anti-PF4 antibodies (analyte) present in the sample. As for the HemosIL® AcuStar HIT-IgG_{(PF4-H)}, the signal (RLU) was directly proportional to the analyte concentration of the sample in the VITT assay.

### 4. Results: Performance of the Reagent of the Disclosure for Detecting VITT

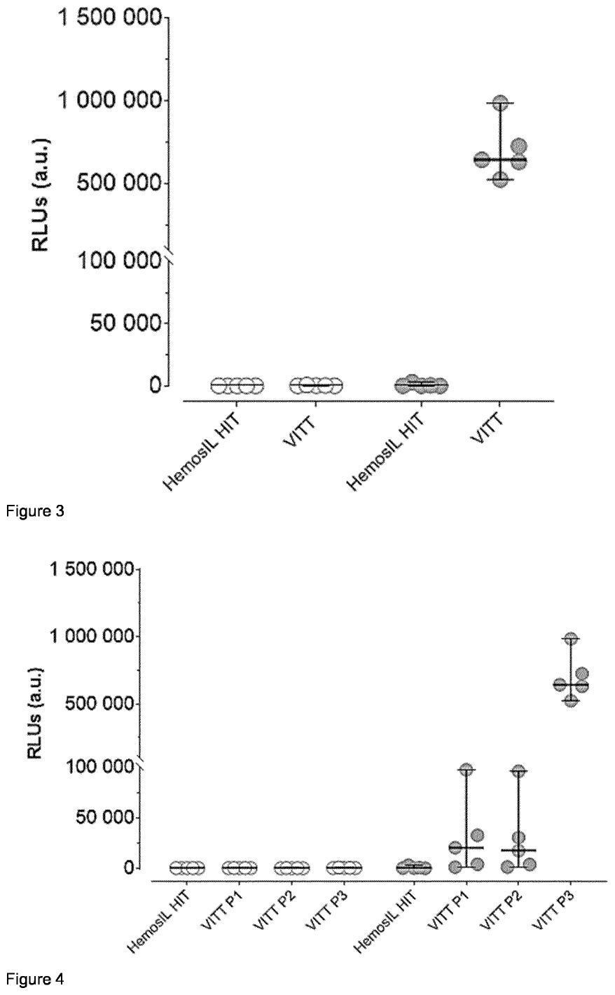

As shown in FIG. 2, the reagent of the disclosure (VITT P3), where PF4 protein alone was covalently bound to the magnetic particles, accurately detected anti-PF4 antibodies in all VITT positive plasmas but it barely detected antibodies in samples from patients with HIT. Similarly, no reactivity was observed with plasma samples from healthy donors. A cutoff line (dashed line) could be established to determine when the VITT assay would be positive for VITT samples only. These results indicate that the VITT assay, which is based in the reagent of the disclosure, is highly specific for VITT detection and can differentiate between VITT and HIT. Moreover, as expected, HemosIL® Acustar HIT-IgG_{(PF4/H)} assay, which uses PF4/PVS complex on magnetic particles, could not detect the vaccine-induced anti-PF4 antibodies in the VITT samples. This is shown in FIG. 3, where it can also be seen that plasma from all VITT patients showed high RLU response only when tested using VITT P3.

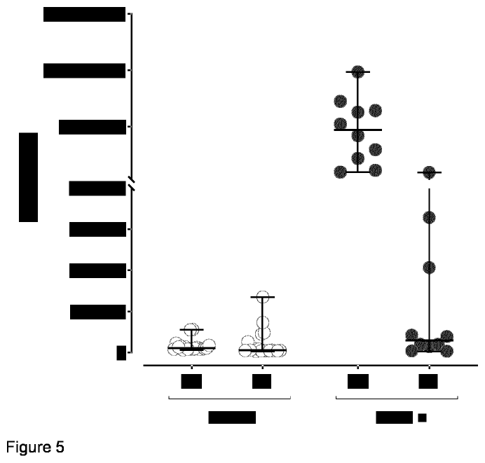

It was also found that covalent coupling of PF4 to the particles was essential for detecting anti-PF4 antibodies in all VITT samples. This is evidenced by the results shown in FIG. 4, where it may be observed that plasma from all VITT patients showed high RLU response when tested using VITT P3 (covalent coupling of the PF4 protein to the—carboxylated—magnetic particles), while conversely, VITT P1 and VITT P2 (adsorption of the PF4 protein to the carboxylated magnetic particles) only recognize one of the tested VITT patient samples. Again, none of the VITT plasmas was recognized by the HemosIL® Acustar HIT-IgG_{(PF4/H)} assay. These results were confirmed by comparing results of assays performed with the VITT P3 particles and VITT P1 particles on plasma samples from a higher number of suspected VITT patients (FIG. 5).

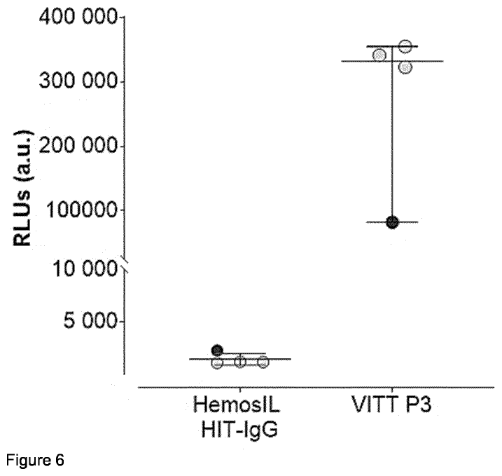

FIG. 6 shows two plasma samples from patients with thrombotic complications of unclear origin (HIT and VITT having been disregarded) which were analyzed with the HemosIL® Acustar HIT IgG(PF4/H) (HemosIL® HIT) and the VITT P3 assay/prototype. As can be seen from FIG. 6, the HemosIL® Acustar HIT IgG assay did not detect anti-PF4 antibodies in these samples, while the VITT assay accurately detected anti-PF4 antibodies in the two plasma samples.

### 5. Performance of the Reagent of the Disclosure for Detecting Other Thrombotic Events not Induced by Heparin

FIG. 7 shows the results of six patients with VITT-like pattern (thrombocytopenia, thrombosis, strongly positive anti-PF4/heparin IgG EIA and PIPA), but without previous exposure to heparin or vaccination in the HemosIL® AcuStar HIT-IgG_{(PF4-H)} assay and the new VITT P3 assay/prototype. Three patients have antibodies anti-PF4 heparin-dependent and anti-PF4 heparin-independent antibodies. The other three of these patients feature anti-PF4 heparin-independent antibodies alone. The results are highly relevant as these patients may benefit of additional treatment with IVIG.

6. Analysis of Sera from Patients with Autoimmune HIT

Acustar HIT-IgG_{(PF4/H)} and the VITT P3 assay/prototype were used to analyse sera from patients clinically suspected of suffering from aHIT. The results showed that 63% of aHIT patients have anti-PF4 antibodies (FIG. 8).

### 7. Conclusions

VITT-like anti-PF4 antibodies are related to severe immunothrombosis independent of heparin. Most of these patients remained previously undiagnosed due to negative results in rapid HIT-tests and heparin-dependent functional tests. The method of the present disclosure allows to identify these patients (also within prospective clinical studies), differentiate HIT- and VITT-like antibodies, and enable rapid clinical decision making.

### CITATION LIST

Greinacher et al, “Thrombotic Thrombocytopenia after ChAdOx1 nCov-19 Vaccination”. DOI: 10.1056/NEJMoa2104840.
Platton et al, “Evaluation of laboratory assays for anti-platelet factor 4 antibodies after ChAdOx1 nCOV-19 vaccination”. DOI: 10.1111/jth. 15362.
Favaloro et al, 2022 “Antibodies against Platelet Factor 4 and Their Associated Pathologies: From HIT/HITT to Spontaneous HIT-Like Syndrome, to COVID-19, to VITT/TTS” DOI: 10.3390/antib1101000
Di Marco M, Shamsuddin S, Razak KA, et al. Overview of the main methods used to combine proteins with nanosystems: absorption, bioconjugation, and encapsulation. Int J Nanomedicine. 2010; 5:37-49.
Biju V. Chem. Soc. Rev., 2014,43, 744-764 (DOI: 10.1039/C3CS60273G)
Huynh, A. et al. Nature volume 596, pages 565-569 (2021) (DOI: 10.1038/s41586-021-03744-4)

1-19. (canceled)

20. An in vitro method for detecting anti-PF4 antibodies comprising:
(i) contacting whole blood, plasma or serum sample obtained from a subject with a reagent comprising: (a) a binding molecule selected from the group consisting of platelet factor 4 protein (PF4), a fragment of PF4 which can bind anti-PF4 antibodies, and an anti-idiotype antibody of anti-platelet factor 4 antibodies (anti-PF4 antibodies); and (b) a solid support, wherein the binding molecule (a) is covalently bound to the surface of the solid support (b), and wherein the binding molecule (a) does not include heparin or a heparin surrogate, and
(ii) analyzing the sample to detect a complex formed by the reagent and the anti-PF4 antibodies;
wherein detecting the complex is indicative of the sample containing anti-PF4 antibodies.

21. The in vitro method of claim 20, wherein detecting the complex is indicative of a thrombotic event not induced by heparin.

22. The in vitro method of claim 21, wherein the thrombotic event not induced by heparin is selected from VITT and a thrombotic event not induced by vaccination.

23. The in vitro method of claim 21, that is for the differential diagnosis of thrombotic events not induced by heparin and heparin-induced thrombotic thrombocytopenia (HIT).

24. The in vitro method of claim 21, further comprising determining the absence of anti-PF4 heparin-dependent antibodies (anti-PF4/H) in the sample.

25. The in vitro method of claim 20, further comprising contacting the mixture obtained from (i) with a tracer capable of binding to the anti-PF4 antibodies.

26. The in vitro method of claim 25, wherein the tracer comprises a labelled anti-human IgG antibody.

27. The in vitro method of claim 20, wherein the reagent consists essentially of (a) a binding molecule selected from the group consisting of PF4, a fragment of PF4 which can bind anti-PF4 antibodies, and an anti-idiotype antibody of anti-platelet factor 4 antibodies (anti-PF4 antibodies); and (b) a solid support; and
wherein the binding molecule (a) is covalently bound to the surface of the solid support (b), and wherein the binding molecule (a) does not include heparin or a heparin surrogate.

28. The in vitro method of claim 20, wherein the binding molecule is PF4.

29. The in vitro method of claim 28, wherein the binding molecule is human PF4.

30. The in vitro method of claim 20, wherein the PF4 is bound to the solid support via an amide bond.

31. The in vitro method of claim 20, wherein the reagent has the following structure: 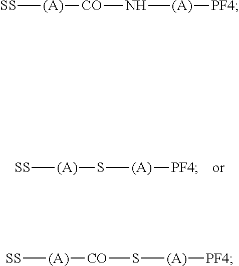
and
wherein SS is the solid support, A is a spacer which can be present or absent from the reagent, and PF4 is the binding molecule capable of binding anti-PF4 antibodies.

32. The in vitro method of claim 20, wherein the solid support is a particle, the particle comprising a microparticle or nanoparticle.

33. The in vitro method of claim 20, wherein the solid support is a microparticle or nanoparticle.

34. The in vitro method of claim 20, wherein the solid support comprises a magnetic metal.

35. A method for recommending or initiating a medical regimen of a subject suspected of suffering a thrombotic event, the method comprising
(a) detecting human anti-PF4 antibodies in a biological sample obtained from the subject by the method of claim 20; and
(b) recommending or initiating an appropriate medical regimen if human anti-PF4 antibodies are detected.

36. The method of claim 35, wherein the medical regime comprises measures to deescalate the FcγRIIa-dependent cell activation that triggers massive hypercoagulability, the measures comprising administering intravenous immunoglobulin (IVIG).

37. The method of claim 20, further comprising detecting anti-PF4/H antibodies in the patient's sample, wherein:
(a) when the sample is positive for anti-PF4 antibodies and negative for anti-PF4/H antibodies, heparin may be administered; or
(b) when the sample is positive for anti-PF4/H antibodies, heparin is discontinued.

38. A kit or a cartridge comprising:
(i) a reagent comprising: (a) a binding molecule selected from the group consisting of platelet factor 4 protein (PF4), a fragment of PF4 which can bind anti-PF4 antibodies, and an anti-idiotype antibody of anti-platelet factor 4 antibodies (anti-PF4 antibodies); and
(b) a solid support, wherein the binding molecule (a) is covalently bound to the surface of the solid support (b), and wherein the binding molecule (a) does not include heparin or a heparin surrogate;
(ii) a further component selected from a tracer capable of binding to the anti-PF4 antibodies, an assay buffer, a diluent, and combinations thereof; and
(iii) instructions for use in detecting anti-PF4 antibodies, or in diagnosing thrombotic events not induced by heparin, or in recommending or initiating a medical regimen of a subject suspicious of suffering a thrombotic event, by a method as defined in any one of the preceding claims.

39. The kit or cartridge of claim 38, wherein the solid support of the reagent is a particle.
